(19) United States
(12) Patent Application Publication
(10) Pub. No.: US 2026/0071047 A1
(43) Pub. Date: Mar. 12, 2026

(54) PROCESSES FOR RECOVERING DIALKYL TEREPHTHALATES FROM TETRAMETHYL CYCLOBUTANEDIOL (TMCD)-CONTAINING POLYMERS

(71) Applicant: Eastman Chemical Company, Kingsport, TN (US)

(72) Inventors: Pinguan Zheng, Johnson City, TN (US); Hannah Grace Harris, Kingsport, TN (US)

(73) Assignee: Eastman Chemical Company, Kingsport, TN (US)

(21) Appl. No.: 18/703,829

(22) PCT Filed: Oct. 25, 2022
(86) PCT No.: PCT/US2022/047647

§ 371 Date: Apr. 23, 2024

Related U.S. Application Data
(60) Provisional application No. 63/262,977, filed on Oct. 25, 2021.

(51) Int. Cl. C08J 11/24; C07C 29/128; C07C 29/80; C07C 67/02; C07C 67/03; C07C 67/48; C08G 63/88; C08J 11/16

(52) U.S. Cl. CPC C08J 11/24; C07C 29/1285; C07C 29/80; C07C 67/02; C07C 67/03; C07C 67/48; C08G 63/88; C08J 11/16; C08J 2367/02

(57) ABSTRACT

Processes for recovering dialkyl terephthalates. The processes can include exposing a feedstock composition that includes one or more tetramethylcyclobutanediol (TMCD)-containing polyesters ethylene glycol, methanol, or both under depolymerization conditions thereby providing one or more depolymerization products. The one or more depolymerization products can be exposed to an alcoholysis process to recover dialkyl terephthalate.

### FIELD OF THE INVENTION

The present disclosure relates to processes for recycling TMCD-containing polymers. More particularly, the present disclosure relates to recovering dialkyl terephthalates from feedstocks comprising TMCD-containing polyesters.

### BACKGROUND OF THE INVENTION

Certain conventional systems may utilize glycolysis and/or methanolysis processes in an attempt to recycle polyesters. However, certain conventional glycolysis and/or methanolysis processes may require a substantial amount of resources and energy in order to arrive at suitable products for use in subsequent production processes, e.g., production processes to generate recycled polyesters or other compositions.

### BRIEF SUMMARY OF THE INVENTION

In one aspect, a process for recovering one or more dialkyl terephthalates from a feedstock is provided. The process can include exposing a feedstock composition comprising one or more tetramethylcyclobutanediol (TMCD)-containing polyesters to: i) ethylene glycol (EG), methanol, or both; and ii) a depolymerization catalyst, in a first reaction vessel under depolymerization conditions to provide a first mixture having a first solid component and a first liquid component. The first liquid component can include one or more depolymerization products. The depolymerization conditions can include a temperature of from 150° C. to 260° C., and a pressure of from 1 atm (14.7 psig) to 102 atm (1500 psig), and a time period of from 0.5 hours to 10 hours. The process can also include cooling at least a portion of the first mixture to a temperature below 150° C. The process can also include exposing at least a portion of the first liquid component to an alcohol composition and an alcoholysis catalyst under conditions including a temperature of from 23° C. to 70° C. and a pressure of from 1 atm to 2 atm for a time period of 0.5 hours to 5 hours to provide a second mixture. The second mixture can include one or more dialkyl terephthalates. The process can also include separating at least a portion of the one or more dialkyl terephthalates by solid-liquid separation.

### BRIEF DESCRIPTION OF THE DRAWING

FIG. 1 is an example system for recovering one or more dialkyl terephthalates from a feedstock composition, in accordance with aspects of the present disclosure.

### DETAILED DESCRIPTION OF THE INVENTION

### Overview

The present disclosure may be understood more readily by reference to the following detailed description of certain aspects of the disclosure and working examples. In according with the purpose(s) of this disclosure, certain aspects of the disclosure are described in the Brief Summary of the Invention and are further described herein below. Also, other aspects of the disclosure are described herein.

Aspects herein are directed to processes for recovering one or more dialkyl terephthalates from feedstock compositions comprising TMCD-containing polyesters. As described herein, in certain aspects, an example process can include exposing a feedstock composition comprising TMCD-containing polyesters to ethylene glycol (EG), methanol, or both, under depolymerization conditions to generate one or more depolymerization products, which are then exposed to an alcoholysis process for recovery of dialkyl terephthalate.

As discussed above, certain conventional glycolysis and/or methanolysis processes may require a substantial amount of resources and energy in order to arrive at suitable products for use in subsequent production processes, e.g., production processes to generate recycled polyesters or other compositions.

The processes and systems disclosed herein can alleviate one or more of the above problems. For instance, in certain aspects, the processes disclosed herein can include exposing feedstock compositions comprising TMCD-containing polyesters to depolymerization conditions with one or more glycols and/or methanol to provide one or more depolymerization products. In various aspects, the one or more depolymerization products can include monomers, oligomers, or a combination thereof. In aspects, the one or more depolymerization products can be exposed to alcoholysis conditions resulting in a dialkyl terephthalate product of high yield and purity. As discussed herein, the alcoholysis conditions include a temperature that is reduced compared to certain conventional systems, which reduces the overall energy and resources required. In aspects, as discussed herein, the depolymerization and alcoholysis conditions described herein are substantially milder than certain conventional processes, which results in less ethylene glycol yield loss, e.g., due to fewer side reactions or degradation reactions converting ethylene glycol into various impurities. Further, in certain aspects as discussed further below, glycols present in the resulting alcoholysis liquid component can be separated from at least a portion of the alcohol composition utilized in the alcoholysis, and these recycle glycols can be re-used in subsequent rounds of dialkyl terephthalate recovery, which also reduces resource consumption.

### Feedstock Compositions

As discussed above, the processes described herein relate to recovering one or more dialkyl terephthalates from a feedstock composition. In certain aspects, the feedstock composition can include one or more polyesters. In various aspects, the feedstock composition can include 2,2,4,4-tetramethyl-1,3-cyclobutanediol (TMCD)-containing polyesters. In certain aspects, the feedstock composition can include TMCD-containing polyesters and, optionally, polyethylene terephthalate (PET). In one or more aspects, the feedstock composition can include TMCD-containing polyesters and polyethylene terephthalate (PET). In various aspects, the feedstock composition can also include, or optionally include, additional components discussed herein, e.g., foreign materials.

The term “polyester” can refer to a synthetic polymer prepared by the reaction of one or more difunctional carboxylic acids and/or multifunctional carboxylic acids with one or more difunctional hydroxyl compounds and/or multifunctional hydroxyl compounds. The difunctional carboxylic acid can be a dicarboxylic acid and the difunctional hydroxyl compound can be a dihydric alcohol such as, for example, glycols. Furthermore, as used herein, the term “diacid” or “dicarboxylic acid” includes multifunctional acids, such as branching agents. The term “glycol” or “diol” as used herein, includes, but is not limited to, diols, glycols, and/or multifunctional hydroxyl compounds. The dicarboxylic acid residues may be derived from a dicarboxylic acid monomer or its associated acid halides, esters, salts, anhydrides, or mixtures thereof. As used herein, therefore, the term dicarboxylic acid is intended to include dicarboxylic acids and any derivative of a dicarboxylic acid, including its associated acid halides, esters, halfesters, salts, half-salts, anhydrides, mixed anhydrides, or mixtures thereof, useful in a reaction process with a diol to make polyester. It should be understood that the term “polyester” as used herein also refers to copolyesters.

As used herein, the term “residue(s)” refers to the monomer unit or repeating unit in a polymer, oligomer, or dimer. As a non-limiting example for illustration purposes, a polymer can be made from the condensation of the following monomers: terephthalic acid (“TPA”) and cyclohexyl-1,4-dimethanol (“CHDM”). The condensation reaction results in the loss of water molecules. The residues in the resulting polymer are derived from either terephthalic acid or cyclohexyl-1,4-dimethanol. Below in Formula (I), a non-limiting example of a polyester is provided.

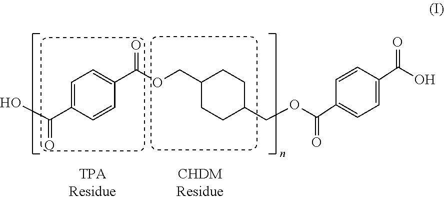

The polyesters for use in the processes disclosed herein typically can be prepared from dicarboxylic acids and diols which react in substantially equal proportions and are incorporated into the polyester polymer as their corresponding residues. The polyesters of the present invention, therefore, can contain substantially equal molar proportions of acid residues (100 mole %) and diol (and/or multifunctional hydroxyl compound) residues (100 mole %) such that the total moles of repeating units is equal to 100 mole %. The mole percentages provided in the present disclosure, therefore, may be based on the total moles of acid residues, the total moles of diol residues, or the total moles of repeating units. For example, a polyester containing 10 mole % isophthalic acid, based on the total acid residues, means the polyester contains 10 mole % isophthalic acid residues out of a total of 100 mole % acid residues. Thus, there are 10 moles of isophthalic acid residues among every 100 moles of acid residues. In another example, a polyester containing 30 mole % TMCD, based on the total diol residues, means the polyester contains 30 mole % TMCD residues out of a total of 100 mole % diol residues. Thus, in such an example, there are 30 moles of TMCD residues among every 100 moles of diol residues

In aspects, the one or more polyesters exhibit an inherent viscosity of from about 0.1 dL/g to about 1.2 dL/g as determined according to ASTM D2857-70, about 0.2 dL/g to about 1.2 dL/g as determined according to ASTM D2857-70, about 0.3 dL/g to about 1.2 dL/g as determined according to ASTM D2857-70, or about 0.4 dL/g to about 1.2 dL/g as determined according to ASTM D2857-70.

In certain aspects, terephthalic acid may be used as the starting material. In another embodiment, dimethyl terephthalate may be used as the starting material. In yet another embodiment, mixtures of terephthalic acid and dimethyl terephthalate may be used as the starting material and/or as an intermediate material. In one or more aspects, the dicarboxylic acid component of the polyesters may include one or more modifying aromatic dicarboxylic acids, such as, for example, isophthalic acid.

As discussed above, in aspects, the feedstock composition can include one or more 2,2,4,4-tetramethyl-1,3-cyclobutanediol (TMCD)-containing polyesters. In certain aspects, any polyesters comprising TMCD are contemplated for use in the processes disclosed herein. In certain aspects, the TMCD-containing polyesters can comprise of from 1 mol % to 60 mol % TMCD, of from 1 mol % to 55 mol % TMCD, of from 1 mol % to 50 mol % TMCD, of from 1 mol % to 40 mol % TMCD, of from 1 mol % to 35 mol % TMCD, of from 1 mol % to 30 mol % TMCD, of from 1 mol % to 25 mol % TMCD, of from 1 mol % to 20 mol % TMCD, or of from 20 mol % to 40 mol % TMCD. In the same or alternative aspects, the TMCD-containing polyesters can comprise 1 mol % to 60 mol % TMCD, of from 1 mol % to 55 mol % TMCD, of from 1 mol % to 50 mol % TMCD, of from 1 mol % to 40 mol % TMCD, of from 1 mol % to 35 mol % TMCD, of from 1 mol % to 30 mol % TMCD, of from 1 mol % to 25 mol % TMCD, of from 1 mol % to 20 mol % TMCD, or of from 20 mol % to 40 mol % TMCD, with the remainder of the diol component being ethylene glycol (EG), CHDM, or both. In certain aspects, the TMCD-containing polyesters can include 1 mol % to 60 mol % TMCD, of from 1 mol % to 55 mol % TMCD, of from 1 mol % to 50 mol % TMCD, of from 1 mol % to 40 mol % TMCD, of from 1 mol % to 35 mol % TMCD, of from 1 mol % to 30 mol % TMCD, of from 1 mol % to 25 mol % TMCD, of from 1 mol % to 20 mol % TMCD, or of from 20 mol % to 40 mol % TMCD; 0 mol % to 99 mol % EG, 1 mol % to 99 mol % EG, 0 mol % to 85 mol % EG, 1 mol % to 85 mol % EG, 40 mol % to 90 mol % EG, or 60 mol % to 80 mol % EG; 0 mol % to 99 mol % CHDM, 1 mol % to 99 mol % CHDM, 0 mol % to 85 mol % CHDM, 1 mol % to 85 mol % CHDM, 40 mol % to 90 mol % CHDM, or 60 mol % to 80 mol % CHDM. In one aspect, the TMCD-containing polyesters can include 20 mol % to 40 mol % TMCD and 60 mol % to 80 mol % EG. In certain aspects, the TMCD-containing polyesters can include 20 mol % to 40 mol % TMCD and 60 mol % to 80 mol % CHDM In certain aspects, the TMCD-containing polyesters can comprise of from 1 mol % to 60 mol % TMCD, of from 1 mol % to 55 mol % TMCD, of from 1 mol % to 50 mol % TMCD, of from 1 mol % to 40 mol % TMCD, of from 1 mol % to 35 mol % TMCD, of from 1 mol % to 30 mol % TMCD, of from 1 mol % to 25 mol % TMCD, of from 1 mol % to 20 mol % TMCD, or of from 20 mol % to 40 mol % TMCD, with the remainder of the diol component being ethylene glycol (EG), CHDM, trimtheylol propane (TMP), mono propylene glycol (MPG), or a combination thereof.

In various aspects, the feedstock composition can include, or can optionally include, polyethylene terephthalate (PET), glycol modified PET, or both. Example glycol modified PETs can include 1,4-cyclohexanedimethanol (CHDM)-modified PET, isophthalic acid (IPA)-modified PET, diethylene glycol (DEG)-modified PET, glycol-modified PET, neopentyl glycol (NPG)-modified PET, propane diol (PDO)-modified PET, butanediol (BDO)-modified PET, heaxanediol (HDO)-modified PET, 2-methyl-2,4-pentanediol (MP diol)-modified PET, isosorbide-modified PET, poly(tetramethylene ether) glycol (PTMG)-modified PET, poly(ethylene) glycol (PEG)-modified PET, polycyclohexylenedimethylene terephthalate (PCT), cyclohexanedimethanol (CHDM)—containing copolyester, isosorbide-containing copolyester, or a combination thereof. In the same or alternative aspect, the polyethylene terephthalate (PET) can comprise CHDM, IPA, DEG, NPG, PDO, BDO, HDO, MP diol, isosorbide, PTMG, PEG, or a combination thereof.

In various aspects, the PET can include CHDM. In one aspect, the PET can include about 0 mole % to about 100 mole % CHDM, about 1 mole % to about 100 mole % CHDM, about 1 mole % to about 90 mole % CHDM, about 1 mole % to about 80 mole % CHDM, about 1 mole % to about 70% CHDM, about 1 mole % to about 60 mole % CHDM, about 1 mole % to about 50 mole % CHDM, about 1 mole % to about 40 mole % CHDM, about 1 mole % to about 35 mole % CHDM, about 1 mole % to about 30 mole % CHDM, about 1 mole % to about 25 mole % CHDM, about 1 mole % to about 20 mole % CHDM, about 1 mole % to about 10 mole % CHDM, or about 1 mole % to about 5 mole % CHDM. In aspects, the mole % of CHDM refers to the mole % of CHDM relative to all diol equivalents in the PET. In various aspects, the PET can include DEG. In aspects, the PET can include about 0 mole % to about 100 mole % DEG, about 1 mole % to about 100 mole % DEG, about 1 mole % to about 90 mole % DEG, about 1 mole % to about 80 mole % DEG, about 1 mole % to about 70 mole % DEG, about 1 mole % to about 60 mole % DEG, about 1 mole % to about 50 mole % DEG, about 1 mole % to about 40 mole % DEG, about 1 mole % to about 35 mole % DEG, about 1 mole % to about 30 mole % DEG, about 1 mole % to about 20 mole % DEG, about 1 mole % to about 10 mole % DEG, about 1 mole % to about 5 mole % DEG, or about 1 mole % to about 3 mole % DEG. In aspects, the mole % of DEG refers to the mole % of DEG relative to all diol equivalents in the PET. In aspects, the PET can include isophthalic acid. In aspects, the PET can include about 0 mole % to about 30 mole % isophthalic acid, about 1 mole % to about 30 mole % isophthalic acid, about 1 mole % to about 25 mole % isophthalic acid, about 1 mole % to about 20 mole % isophthalic acid, about 1 mole % to about 15 mole % isophthalic acid, about 1 mole % to about 10 mole % isophthalic acid, about 1 mole % to about 7.5 mole % isophthalic acid, about 1 mole % to about 5 mole % isophthalic acid, about 1 mole % to about 3 mole % isophthalic acid, about mole % or less of isophthalic acid, about 7.5 mole % or less of isophthalic acid, about 5 mole % or less of isophthalic acid, or about 3 mole % or less of isophthalic acid. In aspects, the mole % of isophthalic acid refers to the mole % of isophthalic acid relative to all diacid equivalents in the PET. In certain aspects, the PET can include about 0 mole % to about 100 mole % CHDM, about 0 mole % to about 100 mole % DEG, about 0 mole % to about 30 mole % isophthalic acid, or a combination thereof. In certain aspects, the PET can include about 1 mole % to about 100 mole % CHDM, about 1 mole % to about 100 mole % DEG, about 1 mole % to about 30 mole % isophthalic acid, or a combination thereof. In various aspects, the PET can include other glycols, e.g., other than those mentioned above. For instance, in aspects, the PET can include, but is not limited to, neopentyl glycol (NPG), 2-methyl-2,4-pentanediol (MP diol), butanediol (BDO), propanediol (PDO), hexanediol (HDO), isosorbide, poly(tetramethylene ether) glycol (PTMG), poly(ethylene) glycol (PEG), or a combination thereof. In certain aspects, each of the NPG, MP diol, BDO, PDO, HDO, isosorbide, PTMG, and PEG can be present in the PET in an amount of 0 mole % to about 100 mole %, about 1 mole % to about 100 mole %, about 1 mole % to about 90 mole %, about 1 mole % to about 80 mole %, about 1 mole % to about 70%, about 1 mole % to about 60 mole %, about 1 mole % to about 50 mole %, about 1 mole % to about 40 mole %, about 1 mole % to about 35 mole %, about 1 mole % to about 30 mole %, about 1 mole % to about 25 mole %, about 1 mole % to about 20 mole %, about 1 mole % to about 10 mole %, or about 1 mole % to about 5 mole %. In aspects, the mole % of each of NPG, MP diol, BDO, PDO, HDO, isosorbide, PTMG, and PEG refers to the mole % of each of NPG, MP diol, BDO, PDO, HDO, isosorbide, PTMG, and PEG, respectively, relative to all diol equivalents in the PET. In various aspects, the PET can include CHDM, DEG, NPG, MP diol, BDO, PDO, HDO, isosorbide, PTMG, PEG, isophthalic acid, or a combination thereof, where each component is present in any of the amounts for such components described in this paragraph.

In various aspects, when PET is present in the feedstock composition, a weight ratio of the PET relative to the TMCD-containing polyester in the feedstock composition can be 10:1 to 1:10, 9:1 to 1:9, 8:1 to 1:8, 7:1 to 1:7, 6:1 to 1:6, 5:1 to 1:5, 4:1 to 1:4, 3:1 to 1:3, or 2:1 to 1:2. In aspects, where a glycol-modified PET is present in the feedstock composition it can be present in the same relative amounts described in this paragraph.

In aspects, the feedstock composition can include recycled polyesters, e.g., recycled TMCD-containing polyesters, recycled PET, or both. In various aspects, the recycled polyester(s) can include material that was recovered as manufacturing scrap, industrial waste, post-consumer waste, or a combination thereof. In aspects, the recycled polyester(s) can be prior-used products that have been used and/or discarded. In aspects, the feedstock composition and/or recycled polyester(s) can come from various sources and/or in various forms, including but not limited to textiles, carpet, thermoformed materials, bottles, pellets, and film. In one or more aspects, the feedstock composition can include renewal polyesters, e.g., renewal TMCD-containing polyesters, renewal PET, or both, formed from DMT recovered from a prior DMT recovery process, such as the processes described herein.

In various aspects, the feedstock composition can include one or more foreign materials. In aspects, the one or more foreign materials may include, but are not limited to, polyesters other than TMCD-containing polyesters and polyethylene terephthalate (PET), polyvinyl chloride (PVC), polyvinyl acetal, polyvinyl butyral (PVB), polyvinyl alcohol (PVOH), ethylene vinyl alcohol (EVOH), cotton, polyolefins, polyethylene, polypropylene, polystyrene, polycarbonate, Spandex, natural fibers, cellulose ester, polyacrylates, polymethacrylate, polyamides, nylon, poly(lactic acid), polydimethylsiloxane, polysilane, calcium carbonate, titanium dioxide, inorganic fillers, dyes, pigments, color toners, colorants, plasticizers, adhesives, flame retardants, metals, aluminum, and iron, or a combination thereof. In various aspects, the one or more foreign materials can be present in the feedstock composition in an amount of from about 0.01 wt. % to about 50 wt. %, about 0.01 wt. % to about 40 wt. %, about 0.01 wt. % to about 30 wt. %, about 0.01 wt. % to about 20 wt. %, about 0.01 wt. % to about 15 wt. %, about 0.01 wt. % to about 10 wt. %, about 0.01 wt. % to about 7.5 wt. %, about 0.01 wt. % to about 5 wt. %, about 0.01 wt. % to about 2.5 wt. %, about 0.01 wt. % to about 1.0 wt. %, relative to the weight of the one or more TMCD-containing polyesters in the feedstock composition, and/or relative to the weight of the TMCD-containing polyesters and PET in the feedstock composition.

In aspects, the feedstock composition can be in solid form, liquid form, molten form, or in a solution. In certain aspects, the solution can include a feedstock composition pre-dissolved in a solvent, e.g., DMT, EG, DEG, TEG, or a combination thereof.

### Optional Pre-treatment of the Polyester Composition

In certain aspects, an optional pre-treatment of the feedstock composition, prior to depolymerization and/or methanolysis, can be performed. In various aspects, the optional pre-treatment can include any type of treatment that aids in removing a portion of any foreign materials from the feedstock composition and/or that aids in recovering one or more polyesters from a mixed feedstock, e.g., a feedstock comprising the foreign materials discussed above. For instance, in one aspect, the optional pre-treatment can include exposing the feedstock composition to one or more solvents, in an effort to selectively dissolve one or more polyesters in the feedstock composition (or at least a portion of the foreign materials in the feedstock composition) to allow for separation between at least a portion of the foreign materials and the TMCD-containing polyesters and/or PET in the feedstock composition. As one example aspect, the optional pre-treatment can include exposing the feedstock composition to one or more solvents, e.g., one or more solvents that can cause dissolution of one or more polyesters in the feedstock composition. For instance, the one or more solvents can include but are not limited to 4-methylcyclohexanemethanol (MCHM), ethylene glycol (EG), diethylene glycol (DEG), triethylene glycol (TEG), 1,4-cyclohexanedimethanol (CHDM), poly(ethylene glycol) (PEG), neopentyl glycol (NPG), propane diol (PDO), butanediol (BDO), 2-methyl-2,4-pentanediol (MP diol), poly(tetramethylene ether) glycol (PTMG), dibutyl terephthalate (DBT), dioctyl terephthalate (DOTP), ethylene carbonate (EC), dimethyl carbonate (DMC), dimethyl sulfoxide (DMSO), dimethylformamide (DMF), or combinations thereof. In the same or alternative aspects, the feedstock composition can be exposed to the one or more solvents at specific temperatures to effectuate dissolution of one or more components. In various aspects, a pre-treatment process can include one or more dissolution and separation steps using various solvents and/or temperatures to achieve a desired level of foreign materials removal and/or purity level of the TMCD-containing polyesters and/or PET. For instance, in one aspect, a dissolution and separation can be utilized using one solvent at a specific temperature, e.g., to remove one or more foreign materials, followed by a subsequent dissolution and separation of a polyester fraction using another solvent at a specific temperature, e.g., to remove one or more other foreign materials. The dissolution and/or separation(s) in this optional pre-treatment step can utilize any suitable systems, reactors, vessels, and/or separation techniques to achieve a desired pre-treated feedstock composition.

### Depolymerization of the Feedstock Composition

As discussed above, in various aspects, the processes disclosed herein can include exposing a feedstock composition to depolymerization conditions to depolymerize at least a portion of the one or more polyesters, e.g., TMCD-containing polyesters and/or PET into one or more depolymerization products. In various aspects, the one or more depolymerization products can include monomers, oligomers, or a combination thereof. In certain aspects, the oligomers can exhibit a degree of polymerization from 2 to 10, 2 to 8, 2 to 6, or 2 to 4. In aspects, the one or more polyesters may be depolymerized into one or more depolymerization products that can include monomers and oligomers having a degree of polymerization from 2 to 10, 2 to 8, 2 to 6, or 2 to 4. In aspects, liquid chromatography can be utilized to discern the degree of polymerization of an oligomer, and/or gel permeation chromatography can be utilized to discern the molecular weight of the oligomers.

In aspects, the term degree of polymerization (DP) can refer to the number of residues in the oligomer. As used herein, the degree of polymerization (DP) refers to the number of difunctional carboxylic acid residues and/or multifunctional carboxylic acid residues in the oligomer. For instance, in one example aspect, a DP of one, would refer to a residue that includes one terephthalic acid residue or one isophthalic acid residue. In such an example aspect, a DP of one can also be referred to as a monomer. A non-limiting example of a DP of one is provided below in formula (II), wherein R can be a glycol, e.g., any of the glycols described herein, in aspects. For instance, for the TMCD-containing polyesters described herein, the R can be TMCD.

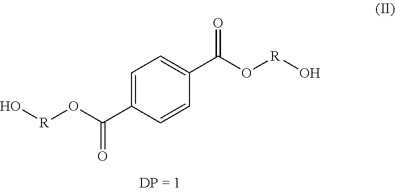

Formulas (III)-(V) below show non-limiting examples of oligomers having a DP of two, three, and n, respectively, in aspects. In Formulas (III), (IV), and (V), R can be a glycol, e.g., any of the glycols described herein, in aspects. For instance, for the TMCD-containing polyesters described herein, the R can be 5 TMCD.

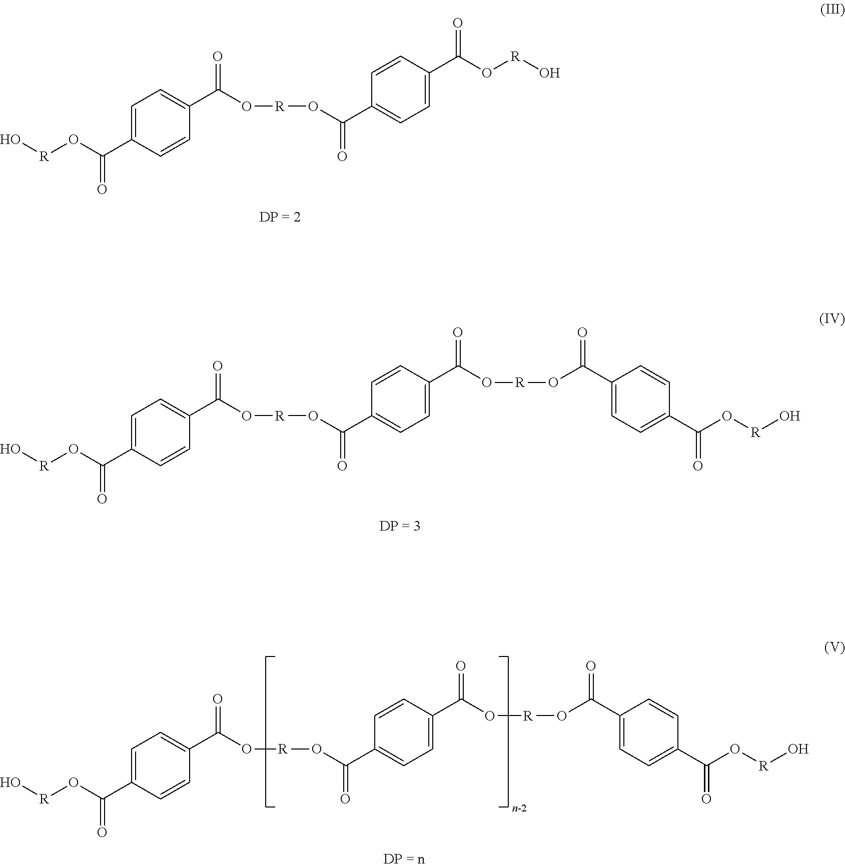

In one or more aspects, this depolymerization process can occur via a glycolysis process. Generally, in aspects, the glycolysis process can include exposing a feedstock composition to one or more glycols, where the glycols react with the polyester, optionally in the presence of a trans-esterification catalyst, forming a mixture of bis(hydroxyethyl) terephthalate (BHET) and low molecular weight terephthalate oligomers. Some representative examples of glycolysis methods are disclosed in U.S. Pat. Nos. 3,257,335; 3,907,868; 6,706,843; and 7,462,649, and are incorporated by reference herein. In certain aspects, the depolymerization process can include exposing the feedstock composition to one or more glycols, methanol, or both under depolymerization conditions.

In one aspect of a depolymerization process, one or more polyesters, e.g., one or more TMCD-containing polyesters, and one or more glycols and/or methanol can be fed into a depolymerization reactor where the TMCD-containing polyesters are dissolved and depolymerized under depolymerization conditions.

In aspects, any amount of the one or more glycols and methanol suitable for use in a depolymerization process can be utilized. In various aspects, the weight ratio of the one or more glycols, methanol, or both relative to the TMCD-containing polyesters in the feedstock composition can be in a range of 10:1 to 2:1, 9:1 to 2:1, 8:1 to 2:1, 7:1 to 2:1, 6:1 to 2:1, 5:1 to 2:1, or 4:1 to 2:1.

In certain aspects, the one or more glycols can include any glycol suitable for use in a glycolysis process. In aspects, the word glycol can refer to aliphatic, alicyclic, and aralkyl glycols. Exemplary glycols include ethylene glycol, 1,2-propandiol (also known propylene glycol), 1,3-propanediol, 1,4-butanediol, 1,5-pentanediol, 1,6-hexanediol, 2,2-dimethyl-1,3-propanediol, 1,2-cyclohexane dimethanol, 1,3-cyclohexane dimethanol, 1,4-cyclohexane dimethanol, 2,2,4,4-tetramethyl-1,3-cyclobutanediol, isosorbide, p-xylylenediol, and the like. These glycols may also contain ether linkages, such as is the case in, for example, diethylene glycol, triethylene glycol, and tetraethylene glycol. Additional embodiments of glycols include higher molecular weight homologs, known as polyethylene glycols, such as those produced by Dow Chemical Company under the Carbowax™ tradename. In one embodiment, the polyethylene glycol has a molecular weight of from greater than 200 to about 10,000 Daltons (Mn). These glycols also include higher order alkyl analogs, such as dipropylene glycol, dibutylene glycol, and the like. Similarly, further glycols include higher order polyalkylene ether diols, such as polypropylene glycol and polytetramethylene glycol with molecular weights ranging from about 200 to about 10,000 Daltons (Mn) (also referred to as g/mole). In one aspect, the glycol can be chosen from aliphatic, alicyclic, and aralkyl glycols. In the same or alternative aspects, the glycol can be chosen from ethylene glycol; 1,2-propandiol; 1,3-propanediol; 1,4-butanediol; 1,5-pentanediol; 1,6-hexanediol; 2,2-dimethyl-1,3-propanediol; 1,2-cyclohexane dimethanol; 1,3-cyclohexane dimethanol; 1,4-cyclohexane dimethanol; 2,2,4,4-tetramethyl-1,3-cyclobutanediol; isosorbide; p-xylylenediol; diethylene glycol; triethylene glycol; tetraethylene glycol; polyethylene glycols; dipropylene glycol; dibutylene glycol; polyalkylene ether diols chosen from polypropylene glycol and polytetramethylene glycol.

In various aspects, the one or more glycols can include ethylene glycol (EG). In certain aspect, EG and methanol can be utilized in the depolymerization process. In certain aspects, as discussed in detail below, the one or more glycols can be recycle glycols that were recovered from a prior glycolysis and methanolysis process for recovery of one or more dialkyl terephthalates, as disclosed herein. In the same or alternative aspects, the methanol, when present in the depolymerization process, can be recycled from a glycolysis process, a methanolysis process, or both.

In various aspects, as discussed above, the depolymerization process can include one or more catalysts, e.g., trans-esterification catalysts. In certain aspects, the catalyst can be present in an amount of from 0.1 wt. % to 10 wt. %, relative to the weight of the feedstock composition, or relative to the weight of the TMCD-containing polyesters and/or PET in the feedstock composition. In aspects, any suitable catalyst can be utilized. In one aspect, the catalyst can include a carbonate catalyst, for example, but not limited to: Li₂CO₃, Na₂CO₃, Cs₂CO₃, ZrCO₃, or a combination thereof. In one aspect, the catalyst can include a hydroxide catalyst, for example, but not limited to: LiOH, NaOH, KOH, tetrabutylammonium hydroxide (TBAH), or a combination thereof. In one aspect, the catalyst can include an alkoxide catalyst, for example, but not limited to: sodium methoxide (NaOMe), lithium methoxide (LiOMe), magnesium methoxide, potassium t-butoxide, ethylene glycol monosodium salt, ethylene glycol disodium salt, or a combination thereof. In one aspect, the catalyst can include tetra isopropyl titanate (TIPT), butyltin tris-2-ethylhexanoate (FASCAT 4102), 1,8-Diazabicyclo[5.4.0]undec-7-ene (DBU), zinc acetylacetonate hydrate (Zn(acac)₂), zinc acetate (Zn(OAc)₂), and manganese (II) acetate (Mn(OAc)₂)), or a combination thereof. In certain aspects, the catalyst can include LiOH, NaOH, KOH, tetra isopropyl titanate (TIPT), butyltin tris-2-ethylhexanoate (FASCAT 4102), ZrCO₃, 1,8-Diazabicyclo[5.4.0]undec-7-ene (DBU), sodium methoxide (NaOMe), lithium methoxide (LiOMe), and zinc acetylacetonate hydrate (Zn(acac)₂), or a combination thereof. In one aspect, the catalyst can include LiOH, NaOH, KOH, sodium methoxide (NaOMe), and lithium methoxide (LiOMe). In certain aspects, the catalyst can include Li₂CO₃, CaCO₃, Na₂CO₃, Cs₂CO₃, ZrCO₃, LiOH, NaOH, KOH, tetrabutylammonium hydroxide (TBAH), sodium methoxide (NaOMe), lithium methoxide (LiOMe), magnesium methoxide (Mg(OMe)₂, potassium t-butoxide, ethylene glycol monosodium salt, ethylene glycol disodium salt, tetra isopropyl titanate (TIPT), butyltin tris-2-ethylhexanoate (FASCAT 4102), 1,8-Diazabicyclo[5.4.0]undec-7-ene (DBU), zinc acetylacetonate hydrate (Zn(acac)₂), zinc acetate (Zn(OAc)₂), manganese (II) acetate (Mn(OAc)₂), hydrotalcite, zeolite, lithium chloride, or a combination thereof.

The depolymerization conditions can include a temperature of from 150° C. to 260° C. and an absolute pressure of from 1 atm (14.7 psig) to 102 atm (1500 psig), of from 1 atm (14.7 psig) to 50 atm (734 psig), of from 1 atm (14.7 psig) to 30 atm (440 psig), of from 1 atmosphere (atm) (14.7 psig) to atm (220 psig), or of from 1 atm (14.7 psig) to 2 atm (30 psig), in an agitated reactor for 0.5 h to 10 h. Higher temperatures may be used to increase the rate of depolymerization; however, reactor systems that can withstand elevated pressures may be required. One or a plurality of reactors may be used for the reaction of the polyester with the one or more glycols and/or methanol. For example, the reaction mixture can be continuously withdrawn from the first stage and introduced to a second stage maintained under pressure, along with additional glycol and/or methanol, wherein depolymerization continues to the desired degree of completion. In various aspects, any type of vessel, reactor, and/or reactor system can be utilized for the depolymerization or glycolysis of the polyester. In one aspect, a continuous stirred-tank reactor or vessel, a fixed bed reactor, or a melt extruder. In the same or alternative aspects, the depolymerization or glycolysis of the feedstock composition can be a batch or continuous process.

Upon exposure to the depolymerization conditions detailed above, the resulting mixture can optionally be allowed to cool to a temperature of about 150° C. or less, or of from about 50° C. to about 150° C. In aspects, the resulting mixture can be allowed to cool to the desired temperature in the depolymerization reaction vessel(s) or can be transferred to a different vessel for temperature reduction. The resulting mixture can include a solid component and a liquid component. In aspects, the liquid component includes the one or more depolymerization products, e.g., monomers and/or oligomers having a degree of polymerization of from 2 to 10, along with the one or more glycols and/or methanol, and may also include any additional soluble components from the polyester composition, one or more glycols, catalysts, or a combination thereof. In various aspects, the solid component can be the residual foreign materials and any other insoluble components of the polyester composition and may be considered a waste product to discard.

As discussed further below, the liquid component is further subjected to at least a methanolysis process for the recovery of one or more dialkyl terephthalates. In various aspects, prior to the methanolysis process, the liquid component can be separated from the solid component. In aspects, the liquid component can be separated from the solid component using any system. In one aspect, the liquid component can be separated from the solid component while the resulting mixture is at a temperature of from about 50° C. to about 150° C. In such aspects, separating the liquid component from the solid component at temperatures of about 150° C. or less, e.g., at about 50° C. to about 150° C., can provide for a more efficient process and/or less resource intensive process than current conventional processes. In the same or alternative aspects, separating the liquid component from the solid component at temperatures of about 150° C. or less, e.g., at about 50° C. to about 150° C., can be beneficial as some impurities can be unstable at higher temperatures, e.g., temperatures above 150° C., which may adversely affect the processes, product yields, and/or product purities described herein.

In various aspects, the separation of the liquid component from the solid component can include a filtration process. In such an aspect, any suitable filtration process can be utilized that is capable of withstanding the increased filtration temperatures of from about 50° C. to about 150° C. In certain aspects, the solid component can be removed by centrifugation. In certain aspects, the solid can be removed by settling or sedimentation. In certain aspects, the solid component may have settled in the bottom of a vessel allowing for removal of the liquid component through a vessel conduit or valve appropriately positioned within the vessel. In one aspects, such a conduit and/or valve may include a filtration device to minimize the inclusion of solid component in downstream processes.

In one or more aspects, the foreign materials and/or one or more solid components resulting from the depolymerization process may not be separated from the depolymerization products prior to methanolysis, and instead, may be separated during the isolation of the dialkyl terephthalate resulting from the methanolysis process.

### Alcoholysis of the One or More Depolymerization Products

As discussed above, in aspects, the one or more depolymerization products produced in the depolymerization process described above can be subjected to an alcoholysis process.

Generally, in a typical alcoholysis process, a polyester is reacted with an alcohol, e.g., methanol, to produce a depolymerized mixture comprising oligomers, terephthalate monomers, e.g., dimethyl terephthalate (DMT), and one or more glycols. In other embodiments, other monomers such as, for example, CHDM, DEG, and dimethyl isophthalate (DMI), also may be produced, depending on the composition of the polyester. In one embodiment, during the alcoholysis process the terephthalate oligomers are reacted with methanol to produce a depolymerized polyester mixture comprising polyester oligomers, DMT, CHDM, and/or EG.

Some representative examples of the methanolysis of PET are described in U.S. Pat. Nos. 3,321,510; 3,776,945; 5,051,528; 5,298,530; 5,414,022; 5,432,203; 5,576,456; 6,262,294; which are incorporated herein by reference.

In aspects, the alcoholysis process can include exposing the liquid component and/or the one or more depolymerization products resulting from the depolymerization process to an alcohol composition under conditions resulting in one or more dialkyl terephthalates. As discussed above, in aspects, the one or more depolymerization products can be present in the liquid component resulting from the depolymerization process. In various aspects, as discussed above, prior to subjecting the one or more depolymerization products and/or the liquid component resulting from the depolymerization process to an alcoholysis process, the liquid component may be separated from the resulting mixture and/or from the resulting solid component of the glycolysis process. In certain aspects, after separating the liquid component from the solid component of the depolymerization process, the liquid component can be directly utilized in this alcoholysis process. In the same or alternative aspects, after separating the liquid component from the solid component of the glycolysis process, the liquid component is not subjected to any further processing, e.g., distillation and/or other separation processes, prior to being utilized in this alcoholysis process.

The alcohol composition can include any suitable alcohol known in the art for use in an alcoholysis process to obtain a specific dialkyl terephthalate. In one aspect, the alcohol composition can be methanol. In aspects, when methanol is utilized as the alcohol composition, DMT can be the resulting methanolysis product.

In certain aspects, the amount of the alcohol composition can be any amount that is in excess on a weight basis relative to the amount or weight of the feedstock composition and/or of the TMCD-containing polyesters in the feedstock composition. In certain aspects, a weight ratio of the amount of the alcohol composition relative to the amount of the feedstock composition and/or of the TMCD-containing polyesters in the feedstock composition can be from about 2:1 to about 10:1. In such aspects, the amount of the feedstock composition and/or of the TMCD-containing polyesters in the feedstock composition refers to the amount or weight of the feedstock composition and/or of the TMCD-containing polyesters in the feedstock composition that is utilized in the above depolymerization process.

In aspects, the alcoholysis reaction can occur at a temperature of about 90° C. or less, about 80° C. or less, about 70° C. or less, about 60° C. or less, about 50° C. or less, about 40° C. or less, or about 30° C. or less. In the same or alternative aspects, the alcoholysis reaction can occur at a temperature of from about 20° C. to about 90° C., about 20° C. to about 80° C., about 20° C. to about 70° C., about 20° C. to about 60° C., about 20° C. to about 50° C., about 20° C. to about 40° C., about 20° C. to about 30° C., or about 23° C. to about 70° C. In various aspects, without being bound by any particular theory, it is believed that, since in the processes disclosed herein, the TMCD-containing polyester and/or PET in the feedstock composition has already undergone at least a partial depolymerization process, e.g., in the depolymerization step discussed above, that the methanolysis process can be performed at the temperatures described above, which are comparably reduced compared to certain other conventional processes. Additionally or alternatively, without being bound by any particular theory, it is believed that, since the one or more depolymerization products produced in the depolymerization process may be separated from the waste or insoluble material prior to this alcoholysis process, the alcoholysis process can be conducted at the reduced temperatures described above, in certain aspects.

In aspects, the alcoholysis process can be conducted in any suitable reactor and/or vessel. In aspects, the alcoholysis reactor can be in fluid communication with the reactor utilized in the depolymerization process described above. In certain aspects, the alcoholysis reactor is a different reactor than the vessel used for depolymerization. Alternatively, in various aspects, the alcoholysis process can be conducted in the same vessel as the depolymerization process and/or the filtration process discussed above. In certain aspects, the alcoholysis process can be conducted at ambient pressure, e.g., about 1 atm, or at a pressure of from about 1 atm to about 5 atm, of from about 1 atm to about 3 atm, or of from about 1 atm to about 2 atm. In various aspects, the alcoholysis reaction can be conducted at a pressure above ambient pressure, e.g., more than 1 atm, or about 5 atm or less, about 3 atm or less, or about 2 atm or less, when the alcoholysis reaction temperature is high for the process conditions disclosed herein, e.g., about 50° C. or more, about 60° C. or more, about 70° C. or more, about 80° C. or more, or about 90° C. or more.

In various aspects, an alcoholysis catalyst can be utilized in the alcoholysis process. In aspects, the alcoholysis catalyst can be present in an amount of from about 0.1 wt. % to about 20 wt. % relative to the weight of the feedstock composition and/or of the TMCD-containing polyesters in the feedstock composition, or of from about 0.1 wt. % to about 10 wt. % relative to the weight of the feedstock composition and/or of the TMCD-containing polyesters in the feedstock composition, or of from about 0.1 wt. % to about 5 wt. % relative to the weight of the feedstock composition and/or of the TMCD-containing polyesters in the feedstock composition, or of from about 0.1 wt. % to about 2 wt. % relative to the weight of the feedstock composition and/or of the TMCD-containing polyesters in the feedstock composition, or of from about 0.1 wt. % to about 1 wt. % relative to the weight of the feedstock composition and/or of the TMCD-containing polyesters in the feedstock composition, or of from about 0.1 wt. % to about 0.5 wt. % relative to the weight of the feedstock composition and/or of the TMCD-containing polyesters in the feedstock composition. In such aspects, the amount of the feedstock composition and/or of the TMCD-containing polyesters in the feedstock composition refers to the amount or weight of the feedstock composition and/or of the TMCD-containing polyesters in the feedstock composition that is utilized in the above depolymerization process. In various aspects, the alcoholysis catalyst amounts disclosed in this paragraph refer to the amount of alcoholysis catalyst present during the alcoholysis reaction. In various aspects, the alcoholysis catalyst amounts disclosed in this paragraph refer to the amount of alcoholysis catalysts that is added to one or more depolymerization products and the one or more alcohols to facilitate the alcoholysis reaction. In certain aspects, reduced or lower amounts of alcoholysis catalyst may be added to the one or more depolymerization products and the one or more alcohols to facilitate the alcoholysis reaction, such as an amount of from about 0.1 wt. % to about 10 wt. % relative to the weight of the feedstock composition and/or of the TMCD-containing polyesters in the feedstock composition, or of from about 0.1 wt. % to about 5 wt. % relative to the weight of the feedstock composition and/or of the TMCD-containing polyesters in the feedstock composition, or of from about 0.1 wt. % to about 2 wt. % relative to the weight of the feedstock composition and/or of the TMCD-containing polyesters in the feedstock composition, or of from about 0.1 wt. % to about 1 wt. % relative to the weight of the feedstock composition and/or of the TMCD-containing polyesters in the feedstock composition, or of from about 0.1 wt. % to about 0.5 wt. % relative to the weight of the feedstock composition and/or of the TMCD-containing polyesters in the feedstock composition. In aspects, such a lower amount of alcoholysis catalysts may be added at least partly because alcoholysis catalyst is already present in the one or more depolymerization products and/or one or more alcohols. In such aspects, as discussed below, the alcohol and/or glycol may be recycled and re-used in subsequent depolymerization and alcoholysis process as disclosed herein, which may include at least a portion of alcoholysis catalyst from a prior alcoholysis and/or glycolysis process.

In various aspects, the alcoholysis catalyst can include a carbonate catalyst, for example, but not limited to: K₂CO₃, Na₂CO₃, Li₂CO₃, Cs₂CO₃; a hydroxide catalyst, for example, but not limited to: KOH, LiOH, NaOH; an alkoxide catalyst, for example, but not limited to NaOMe, Mg(OMe)₂, KOMe, KOt-Bu, ethylene glycol monosodium salt, ethylene glycol disodium salt, or a combination thereof. In certain aspects, the alcoholysis catalyst can include KOH, NaOH, LiOH, or a combination thereof. In certain aspects, the alcoholysis catalyst can include NaOMe, KOMe, Mg(OMe)₂, KOt-Bu, ethylene glycol monosodium salt, ethylene glycol disodium salt, or a combination thereof. In various aspects, the alcoholysis catalyst can be in solid form, a solution form in water, methanol, or ethylene glycol, or a combination of thereof. In certain aspects, the alcoholysis catalyst can be added to the one or more depolymerization products and the alcohol composition once the alcohol composition and the one or more depolymerization products reach the desired reaction temperature or temperature range disclosed above.

The one or more depolymerization products can be exposed to the alcohol composition and optionally the alcoholysis catalyst under the temperature and pressure conditions described above for a period of time to achieve the desired yield of the resulting dialkyl terephthalate. In certain aspects, the one or more depolymerization products can be exposed to the alcohol composition and optionally the alcoholysis catalyst under the temperature and pressure conditions described above for a period of time from about 0.5 hours to about 5 hours.

In aspects, the alcoholysis process results in a mixture that includes one or more dialkyl terephthalates. In various aspects, the alcoholysis process results in mixture wherein the dialkyl terephthalate is an insoluble and/or solid component. In aspects, the liquid component of this mixture can include glycols, the alcohol composition, DEG, CHDM, or a combination thereof. In one aspect, the glycols can be the glycols that were utilized in the depolymerization process and present with the one or more depolymerization products at the initiation of the alcoholysis process. In various aspects, the dialkyl terephthalate can be isolated from the mixture using any known separation technique, e.g., filtering, centrifugation, sedimentation, settling, or a combination of one or more separation techniques. In aspects, the filtering may include washing the solid component with additional alcohol composition or other solvent. The resulting liquid component can include the filtrate and wash. The resulting solid component can include about 90 wt. % or more dialkyl terephthalate, e.g., DMT, about 93 wt. % or more dialkyl terephthalate, e.g., DMT, or about 95 wt. % or more dialkyl terephthalate, e.g., DMT, relative to the weight of the solid component. In the same or alternative aspects, the dialkyl terephthalate, e.g., DMT, in the resulting solid component can be about 90% or more pure, about 93% or more pure, or about 95% or more pure. In various aspects, the solid component can also include dimethyl isophthalate (DMI). In such aspects, the DMI can be present in an amount of about 1000 ppm or less, or about 500 ppm or less, or of from about 1 ppm to about 1000 ppm, or of from about 1 ppm to about 500 ppm. In one or more aspects, the solid component can also include bisphenol A (BPA). In such aspects, the BPA can be present in an amount of about 1000 ppm or less, or about 500 ppm or less, or of from about 1 ppm to about 1000 ppm, or of from about 1 ppm to about 500 ppm.

The processes described herein, e.g., the depolymerizaion and/or alcoholysis processes are substantially mild compared to certain conventional processes, e.g., high temperature one-step glycolysis or methanolysis processes. For instance, certain conventional one-step processes may utilize a glycolysis process at temperatures of 240° C. or above in the presence of a Lewis acid catalyst, for instance, Zn(OAc) 2 or KOAc. Such harsh conditions can result in reduced EG yield from the depolymerization, as the EG is converted in various side reactions to various impurity compounds, including but not limited to: diethylene glycol (DEG), triethylene glycol (TEG), acetaldehyde, 1,1-dimethoxyethane, 1,2-dimethoxyethane, dioxane, 2-methoxyethanol, 1-methoxyethanol, and dimethyl ether. In aspects, the processes described herein are substantially milder than such conventional processes, and also result in less EG yield loss, e.g., from less side reactions converting EG into various impurities. In one aspect, the processes described herein result in about 5% or less yield loss of EG, about 2% or less yield loss of EG, or about 1% or less yield loss of EG, or about 0.5% or less yield loss of EG. In such aspects, the yield loss of EG is the percent of EG that is formed into impurities, e.g., DEG, relative to the combined amount of EG from the polyester composition feed and of the EG added in the glycolysis process. In the same or alternative aspects, the processes described herein result in minimal glycol impurities being produced. For instance, in one aspect, the processes described herein can result in the net generation of about 5 wt. % or less DEG, about 2 wt. % or less DEG, or about 1 wt. % or less DEG, or about 0.5 wt. % or less DEG, or of from about 0.01 wt. % to about 5 wt. % DEG, about 0.01 wt. % to about 2 wt. % DEG, or about 0.01 wt. % to about 1 wt. % DEG, or about 0.01 wt. % to about 0.5 wt. % DEG, or about 0.01 wt. % to about 0.2 wt. % DEG, when EG is used as the one or more glycols in the glycolysis process. In aspects, the processes described herein can result in the net generation of about 5 wt. % or less DEG and/or other impurity, about 2 wt. % or less DEG and/or other impurity, or about 1 wt. % or less DEG and/or other impurity, or about 0.5 wt. % or less DEG and/or other impurity, or of from about 0.01 wt. % to about 5 wt. % DEG and/or other I impurity, about 0.01 wt. % to about 2 wt. % DEG and/or other impurity, or about 0.01 wt. % to about 1 wt. % DEG and/or other impurity, or about 0.01 wt. % to about 0.5 wt. % DEG and/or other impurity, or about 0.01 wt. % to about 0.2 wt. % DEG and/or other impurity, when EG is used as the one or more glycols in the glycolysis process. In aspects, the net generation of DEG (or other impurity) is the weight percent of the amount of DEG or other impurity that is present over the amount of DEG or other impurity present in the polyester composition feed. In one aspect, the DEG being produced can be produced in the glycolysis process described herein and/or the alcoholysis process described herein. In certain aspects, the EG and/or any glycol impurities, such as DEG when using EG as the one or more glycols in the glycolysis process, can be present in the resulting liquid component from this alcoholysis step. In certain aspects, utilization of a Lewis base catalyst, e.g., a hydroxide-based or carbonate-based catalyst, in the glycolysis process may also facilitate or contribute to reduced EG degradation and/or a reduction of glycol impurities. In certain aspects, the process described herein also or alternatively result in reduced yield loss of TMCD. For instance, in various aspects, the processes described herein result in about 30% or less yield loss of TMCD, about 25% or less yield loss of TMCD, about 20% or less of yield loss of TMCD, about 15% or less of yield loss of TMCD, about 10% or less yield loss of TMCD, or about 5% or less of yield loss of TMCD. In the same or alternative aspects, the process described herein also or alternatively result in reduced yield loss of CHDM. For instance, in various aspects, the processes described herein result in about 15% or less yield loss of CHDM, about 10% or less yield loss of CHDM, about 5% or less of yield loss of CHDM, or about 1% or less yield loss of CHDM.

### Recycling Glycols

As discussed above, in various aspects, the glycols utilized in the glycolysis process can be re-used in subsequent rounds of processes for recovery of one or more dialkyl terephthalates disclosed herein. At a high level, in aspects, the liquid component resulting from the alcoholysis process can be processed for re-use, e.g., for re-use in subsequent rounds of glycolysis of a subsequent polyester composition to recover one or more dialkyl terephthalates.

In aspects, as discussed above, the liquid component resulting from the alcoholysis process can include glycols, the alcohol composition, DEG, CHDM, TMCD, or a combination thereof. In certain aspects, the glycols in this liquid component can be the glycols that were utilized in the depolymerization process and present with the one or more depolymerization products at the initiation of the alcoholysis process. In various aspects, this liquid component can be subjected to a separation process, e.g., to remove or separate at least a portion of the alcohol composition, for instance, methanol, or a mixture of methanol and ethylene glycol. In certain aspects, for removal of at least a portion of the alcohol composition, the liquid component can be exposed to distillation or short path distillation. In such aspects, the distillation conditions can include exposing the liquid component to a temperature of about 220° C. or less, about 200° C. or less, about 180° C. or less, about 160° C. or less, about 150° C. or less, about 130° C. or less, about 60° C. or more, about 70° C. or more, of from about 60° C. to about 220° C., of from about 70° C. to about 220° C., of from about 60° C. to about 180° C., or of from about 60° C. to about 160° C. In the same or alternative aspects, the distillation conditions can include a pressure of from about 1 Torr (133.3 Pa) to about 800 Torr (106,657 Pa), or about 30 Torr (3999 Pa) to about 500 Torr (66,661 Pa). In aspects, the liquid component can be exposed to the distillation conditions until all or a substantial portion of the alcohol composition has been removed, e.g., vaporized, from the liquid component. In certain aspects, at least a portion of the alcohol composition, if present with the recycle glycols, may be removed during a subsequent glycolysis process, e.g., may be removed or vaporized due to the glycolysis conditions.

In aspects, the distillation of the liquid component can occur in any vessel or distillation system that is suitable for use in the processes and systems described herein. In one aspect, the distillation vessel can be in fluid communication with the alcoholysis reaction vessel and/or any component of the filtering process utilized subsequent to the alcoholysis, e.g., to isolate the dialkyl terephthalate solid or insoluble component. In the same or alternative aspects, the distillation vessel can be in fluid communication with the glycolysis vessel.

In various aspects, the distillation of the liquid component can cause the alcohol composition to vaporize leaving a pot residue. In aspects, the pot residue includes the glycols and any other heavies, e.g., non-vaporizable compounds present in the liquid component. In aspects, the glycols in the pot residue can be referred to as recycle glycols and/or the glycols from a non-vaporizable portion of a continuous distillation process using the distillation conditions described herein can be referred to as recycle glycols.

In aspects, as discussed above, the recycle glycols can be utilized in a subsequent round of the process described herein to recover one or more dialkyl terephthalates from a polyester composition. Further, in aspects, the recycle glycols can be recycled again using the process described herein, after going through this subsequent round of dialkyl terephthalate recovery. In aspects, the recycle glycols can be recovered and re-used at least two, at least three, at least four, or at least five times. In certain aspects, when the recycle glycols are used in subsequent round(s) of dialkyl terephthalate recovery, addition of a catalyst in the subsequent glycolysis step(s) may be omitted, as the recycle glycols may include prior-used catalyst.

In aspects, when the recycles glycols are recovered and re-used, the resulting dialkyl terephthalates recovered exhibit comparable purity to that of dialkyl terephthalates recovered using glycols that have not been recovered and re-used. This comparable purity of the dialkyl terephthalate is present, in aspects, after re-using recycle glycols at least two times, at least three times, at least four times, or at least five times resulting in a dialkyl terephthalate recovery having a purity of at least about 90%, at least about 93%, or at least about 95%.

As discussed above, in aspects, the processes described herein are substantially milder than certain conventional processes, and also result in less EG yield loss, e.g., due to fewer side reactions converting EG into various impurities. In one example with three EG recycle experiments, the yield loss of EG to DEG is about 5% or less, about 2% or less, about 1% or less, or about 0.5% or less. In one example with four EG recycle experiments, the yield loss of EG to DEG is about 5% or less, about 2% or less, about 1% or less, or about 0.5% or less. In the same or alternative aspects, the processes described herein result in minimal glycol impurities being produced. In certain aspects, the EG and/or any glycol impurities, such as DEG when using EG as the one or more glycols in the glycolysis process, can be present in the resulting liquid component from the alcoholysis process discussed above. In such aspects, the DEG or any glycol impurities can be recovered and/or present in the recycle glycols described herein. In such aspects, the recycle glycols can include about 5 wt. % or less DEG and/or other impurity, about 2 wt. % or less DEG and/or other impurity, or about 1 wt. % or less DEG and/or other impurity, or about 0.5 wt. % or less DEG and/or other impurity, or of from about 0.01 wt. % to about 5 wt. % DEG and/or other impurity, about 0.01 wt. % to about 2 wt. % DEG and/or other impurity, or about 0.01 wt. % to about 1 wt. % DEG and/or other impurity, or about 0.01 wt. % to about 0.5 wt. % DEG and/or other impurity, or about 0.01 wt. % to about 0.2 wt. % DEG and/or other impurity, when EG is used as the one or more glycols in the glycolysis process.

### Use of Recovered Dialkyl Terephthalates to Form Polyesters or Other Products

As discussed above, the processes disclosed herein can result in high purity dialkyl terephthalates, such as DMT. In certain aspects, the recovered DMT can be utilized to form one or more polyesters, including but not limited to PET and TMCD-containing polyesters. In certain aspects, the polyesters formed using recovered DMT can be termed renewal polyesters. In various aspects, the products formed using the recovered DMT may be indistinguishable from similar products formed from virgin DMT. In such aspects, any suitable process for forming the PET and TMCD-containing polyesters can be utilized, since the DMT is of sufficient purity.

In the same or alternative aspects, the recovered DMT can be utilized to form CHDM. In various aspects, the CHDM formed using recovered DMT may be indistinguishable from CHDM formed from virgin DMT, due to the high purity of the recovered DMT. In such aspects, the CHDM can be formed from the recovered DMT using any suitable process.

In various aspects, the recovered DMT can be utilized to form one or more plasticizers. In certain aspects, the plasticizers formed using recovered DMT can include dibutyl terephthalate (DBT) and/or dioctyl terephthalate (DOTP). In various aspects, the DBT and/or DOTP formed using recovered DMT may be indistinguishable from the DBT and/or DOTP, respectively, formed from virgin DMT, due to the high purity of the recovered DMT. In such aspects, the DBT and/or DOTP can be formed from the recovered DMT using any suitable process.

### Example Systems

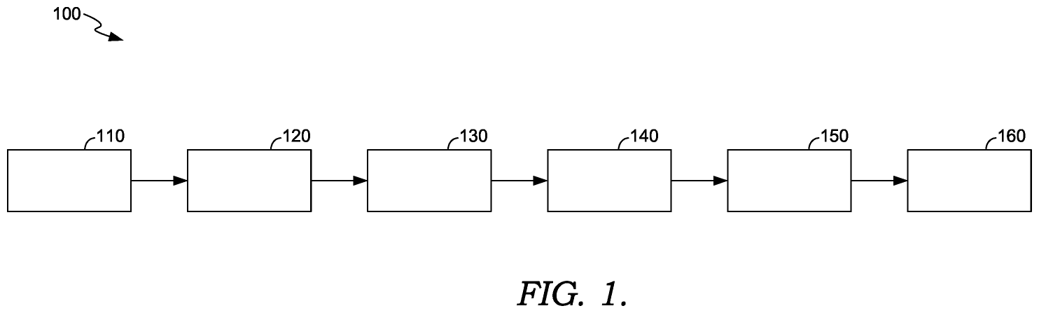

FIG. 1 schematically depicts one example system and/or process for recovering one or more dialkyl terephthalates from a feedstock composition. As discussed above, the feedstock composition comprises one or more TMCD-containing polyesters. The system 100 includes a source 110 of feedstock composition, e.g., the feedstock composition described above. Optionally, as described above, the feedstock composition can be subjected to a pretreatment step to remove at least a portion of the foreign materials prior to entering the depolymerization and alcoholysis process. The vessel 120 represents the depolymerization vessel, where the feedstock composition is received and exposed to one or more glycols and/or methanol under depolymerization conditions, as discussed in detail above. In aspects, the vessel 120 can be in fluid communication with the source 110. In various aspects, as discussed above, the feedstock composition, after exposure to the depolymerization conditions in the vessel 120, is converted into one or more depolymerization products. In various aspects, as discussed above, the one or more depolymerization products can include monomers and/or oligomers having a degree of polymerization of from 2 to 10, 2 to 8, 2 to 6, or 2 to 4. In aspects, the one or more depolymerization products are present in a mixture that includes a liquid component and a solid component, with the one or more depolymerization products in the liquid component. In aspects, as discussed above, this mixture can be exposed to a solid-liquid separation device 130, e.g., a filtering system, where the liquid component, containing the one or more depolymerization products, is separated from the solid component. In various aspects, as discussed herein, the solid-liquid separation device 130 can be in fluid communication with the vessel 120 and/or with the vessel 140. In the aspect depicted in FIG. 1, the one or more depolymerization products and/or the liquid component can be exposed to alcoholysis conditions in a vessel 140. In aspects, the one or more depolymerization products and/or the liquid component can be directly utilized in this alcoholysis process. In such an aspect, the one or more depolymerization products and/or the liquid component may not be subjected to any further processing, e.g., distillation and/or other separation processes, prior to being utilized in this alcoholysis process. Alcoholysis conditions are discussed in detail above. In aspects, as discussed above, the alcoholysis of the one or more depolymerization products and/or the liquid component can result in a mixture that includes an insoluble or solid component that comprises the dialkyl terephthalate and a liquid component that comprises the alcohol composition, glycols, methanol, and/or potentially other soluble components as described herein. As discussed above, the resulting alcoholysis reaction mixture can be exposed to a solid-liquid separation device 150, e.g., a filtering system, to separate the solid component containing the recovered dialkyl terephthalate 160. In aspects, the solid-liquid separation device 150 can be in fluid communication with the vessel 140. In certain aspects, the process described herein associated with the system 100 can be performed as a continuous process, a batch process, or a semi-continuous process. It is understood that the system 100 is just one example system and other configurations of system components are contemplated by the disclosure herein. For instance, one or more of the components of the system 100 may not be physically separated, or distinct, from one or more other components of the system 100. It is further understood that the system 100 is only schematically depicted in order to highlight aspects of the processes disclosed herein.

The present disclosure can be further illustrated by the following examples of aspects thereof, although it will be understood that these examples are included merely for purposes of illustration and are not intended to limit the scope of the disclosure unless otherwise specifically indicated.

### Examples

### Materials

TMCD-containing polymer used in these examples is the commercially available Tritan™ TX1000 copolyester (TX1000), and the Copolyester GMX200 (GMX200), both available from Eastman Chemical, Kingsport, Tennessee.

FDST-5 contains 100 mole % TPA, 93.0 mole % EG, 4.1 mole % CHDM, and 2.9 mole % DEG, and is available from Eastman. IV: 0.751 dL/g.

All other chemicals and reagents are obtained from Aldrich and used as received, unless otherwise mentioned.

### Analytical Procedures

Gas Chromatography (GC) Analysis. GC analysis was performed on an Agilent model 7890B gas chromatograph equipped with a 7693A autosampler and two G4513A towers. The gas chromatograph (GC) was outfitted with two columns—a 60 m×0.32 mm×1.0-micron DB-1701™ (J&W 123-0763) and a 60 m×0.32×1 micron DB-1 ™ (J&W 123-1063)—and samples were injected simultaneously onto both columns. A shared oven temperature program was used, and sample components were detected by flame ionization detection (FID). Five-point calibrations were performed for components of interest. The gas chromatograph was interfaced to an EZChrom Elite Chromatography Data System.

Methanolysis product samples were prepared by adding a known volume of pyridine-based internal standard solution to a known mass of sample and then derivatizing with N,O-Bis(trimethylsilyl)trifluoroacetamide (BSTFA).

$$DMT yield \% was calculated as : ( weight of final DMT ) / ( t heoretical DMT weight ) * 100 \% .$$

DMT GC purity % was calculated as: (weight % of DMT in final product by GC)/(total wt. % by GC)*100%. Major impurities shown in GC include MeOH, water and EG. DMT purity for almost every example is greater than 99% if excluding MeOH, water and EG.

### Examples 1A-1G: Depolymerization Catalysts and EG/Methanol Variations in a Two-Step Process for TX1000

TX1000 was exposed to a two-step process for recovery of DMT, which includes an initial depolymerization step followed by a low temperature methanolysis step. Particularly, in Example 1A-1C and 1E-1G various catalysts and varying amounts of EG/methanol were tested in the initial depolymerization step and then exposed to low temperature methanolysis. Example 1D tested a catalyst in the initial depolymerization step. The results are summarized in Table 1 below.

### Example 1A

A 3-necked 1-liter round-bottom flask was equipped with a mechanic stirrer, a reflux condenser and a thermocouple. TX1000 (70.04 g), butyltin tris(2-ethylhexanoate) (2.1858 g), and ethylene glycol (141.41 g) was charged to the flask. The resulting mixture was heated to 195° C. under nitrogen atmosphere. The reaction was hold at 195° C. for 3 hours until there were no pellets left. The resulting mixture was hazy, white solution, and a sample was taken (1.54 g). The heating mantle was removed, and the solution was allowed to cool to ambient temperature. To the mixture, methanol (197.00 g) was charged. The resulting mixture was heated to reflux (72.6° C.) before 50% NaOH solution (0.204 mL) was added dropwise. Stirring was continued for 30 min. Heating mantle was removed and the flask was allowed to cool to room temperature for a period of 2 hours. The reaction mixture was further cooled in an ice bath. Product was recovered by filtration and dried. Product was obtained as white crystalline solid (5.03 g). GC analysis showed that product was mainly bis(hydroxyethyl) terephthalate (BHET)

### Example 1B

A 3-necked 1-liter round-bottom flask was equipped with a mechanic stirrer, a reflux condenser and a thermocouple. TX1000 (70.10 g), NaOMe (25% in methanol, 2.80 g), Zn(OAc)₂ (0.47 g) and ethylene glycol (139.33 g) was charged to the flask. The resulting mixture was heated to 195° C. under nitrogen atmosphere. The reaction was hold at 195° C. for 3 hours until there were no pellets left. The resulting mixture was hazy, white solution. The heating mantle was removed, and the solution was allowed to cool to ambient temperature. To the mixture, methanol (196.39 g) was charged. The resulting mixture was heated to 50° C. before 50% NaOH solution (0.306 g) was added dropwise. Stirring was continued for 30 min. Heating mantle was removed and the flask was allowed to cool to room temperature for a period of 2 hours. The reaction mixture was further cooled in an ice bath. Product was recovered by filtration and dried. DMT product was obtained as white crystalline solid (43.61 g, 87% yield, 96% GC purity).

### Example 1C

A 3-necked 1-liter round-bottom flask was equipped with a mechanic stirrer, a reflux condenser and a thermocouple. TX1000 (69.43 g), NaOMe, 25% in methanol (2.69 g), and ethylene glycol (138.91 g) was charged to the flask. The resulting mixture was heated to 195° C. under nitrogen atmosphere and held at 195° C. until there were no pellets left. The resulting mixture was hazy white mixture. The heating mantle was removed, and the solution was allowed to cool to ambient temperature. To the mixture, methanol (196.90 g) was charged. The resulting mixture was heated to 50° C. before 50% NaOH solution (0.306 g) was added dropwise. Stirring was continued for 30 min. Heating mantle was removed and the flask was allowed to cool to room temperature for 2 hours. The reaction mixture was further cooled in an ice bath. Product was recovered by filtration and dried. DMT product was obtained as white crystalline solid (40.35 g, 83% yield, 97% GC purity).

### Example 1D

A 3-necked 1-liter round-bottom flask was equipped with a mechanic stirrer, a reflux condenser and a thermocouple. TX1000 (37.49 g), FDST-5 (37.5 g), K₂CO₃ (0.75 g), and ethylene glycol (152.16 g) was charged to the flask. The resulting mixture was heated to 195° C. under nitrogen atmosphere. The reaction was hold at 195° C. for 3 hours. The resulting mixture was hazy, white solution with some remaining pellets. The heating mantle was removed to allow the mixture to cool down. Insoluble solid (22.82 g) was recovered by filtration. Analysis showed that the insoluble solid contained 70.5% Tritan and 29.5% PET. The filtrate (183.65 g) was obtained, which contained 59.0% EG and 20.9% BHET.

### Example 1E

A 100-mL autoclave was equipped with a mechanic stirrer and thermocouple. Pulverized TX1000 (10.01 g), ethylene glycol (16.15 g), methanol (4.0 g), and potassium carbonate (0.07 g). The resulting mixture was heated to 195° C. and hold under 700 psig N2 pressure for 3 hours. The crude mixture was cooled to room temperature before methanol (36.3 g) and 50% sodium hydroxide aqueous solution (0.044 g) were added. The mixture was further heated to 50° C. and hold under 50 psig nitrogen pressure for 30 min. Turn off the heat and slowly cool to room temperature for 2 hours. Product was isolated by filtration and dried. DMT product was obtained as white solid (6.51 g, 81% yield, 99% purity).

### Example 1F

A 100-mL autoclave was equipped with a mechanic stirrer and thermocouple. Pulverized TX1000 (10.01 g), ethylene glycol (4.03 g), methanol (16.0 g), and potassium carbonate (0.07 g). The resulting mixture was heated to 195° C. and hold under 700 psig N2 pressure for 3 hours. The crude mixture was cooled to room temperature before methanol (24.09 g) and 50% sodium hydroxide aqueous solution (0.044 g) were added. The mixture was further heated to 50° C. and hold under 50 psig nitrogen pressure for 30 min. Turn off the heat and slowly cool to room temperature for 2 hours. Product was isolated by filtration and dried. DMT product was obtained as white solid (4.69 g, 66% yield, 99% purity).

### Example 1G

A 100-mL autoclave was equipped with a mechanic stirrer and thermocouple. Pulverized TX1000 (10.02 g), methanol (39.98 g), and potassium carbonate (0.07 g). The resulting mixture was heated to 195° C. and hold under 700 psig N2 pressure for 3 hours. The crude mixture was cooled to room temperature before 50% sodium hydroxide aqueous solution (0.044 g) were added. The mixture was further heated to 50° C. and hold under 50 psig nitrogen pressure for 30 min. Turn off the heat and slowly cool to room temperature for 2 hours. Product was isolated by filtration and dried. DMT product was obtained as white solid (5.89 g, 83% yield, 99% purity).

TABLE 1
DMT yield and DMT Purity Data for Examples 1A-1G

| | | | low temperature | | | |
|---|---|---|---|---|---|---|
| | | | methanolysis | | | |
| | | | | Added | | |
| | Polymer depolymerization | | | MeOH | | |
| | | Tritan/PET/EG/MeOH | | based | DMT | GC |
| | catalyst | (by wt) | catalyst | on PET | (Yield %) | Purity % |
| Example | K2CO3 | 0.5/0.5/2/0 | Do not proceed | | | |
| 1D | | | | | | |
| Example | FASCAT | 1/0/2/0 | NaOH | 2.8 | <5% | None |
| 1A | 4102 | | (1.5 mol %) | | | |
| Example | NaOMe (5 | 1/0/2/0 | NaOH | 2.8 | 87.9% | 96.9% |
| 1B | mol %), | | (1.5 mol %) | | | |
| | Zn(OAc)2 | | | | | |
| | (1 mol %) | | | | | |
| Example | NaOMe (5 | 1/0/2/0 | NaOH | 2.8 | 82.1% | 96.8% |
| 1C | mol %) | | (1.5 mol %) | | | |
| Example | K2CO3 | 1/0/1.6/0.4 | NaOH | 3.6 | 81% | 98.9% |
| 1E | (1.4 mol %) | | (1.5 mol %) | | | |
| Example 1F | K2CO3 | 1/0/0.4/1.6 | NaOH | 2.4 | 66.2% | 99.8% |
| | (1.4 mol %) | | (1.5 mol %) | | | |
| Example | K2CO3 | 1/0/0/4 | NaOH | 0 | 83.0% | 99.8% |
| 1G | (1.4 mol %) | | (1.5 mol %) | | | |

As can be seen in Table 1, in Example 1D, K₂CO₃ catalyst was shown inefficient to depolymerize TMCD-containing polymer; 30% solid was recovered. Similarly, FASCAT 4102 tin catalyst had poor catalyst activity towards the initial depolymerization of TX1000 (Example 1A). A change to NaOMe or NaOMe/Zn(OAc)₂ catalyst was capable of depolymerize TX1000 and turn into DMT product in a 2-step process in 82-87% yield (Examples 1B and 1C).

### Example 1H: Two-Step Depolymerization and Low Temperature Methanolysis of GMX200

A 3-necked 1-liter round-bottom flask was equipped with a mechanic stirrer, a reflux condenser and a thermocouple. GMX200 (75.11 g), K₂CO₃ (0.75 g) and ethylene glycol (150.29 g) was charged to the flask. The resulting mixture was heated to 195° C. under nitrogen atmosphere. The reaction was hold at 195° C. for 3 hours until there were no pellets left. An aliquot was taken and tested. GC analysis showed that this glycolysis product contained 23.7% BHET and 9.6% unknown.

The crude product was allowed to cool to ambient temperature. To the mixture, methanol (299.93 g) was charged. The resulting mixture was heated to 50° C. before 50% NaOH solution (0.468 g) was added dropwise. Stirring was continued for 30 min. Heating mantle was removed and the flask was allowed to cool to room temperature for a period of 2 hours. The reaction mixture was further cooled in an ice bath. Product was recovered by filtration and dried. DMT product was obtained as white crystalline solid (49.12 g, 74.4% yield, 96% GC purity).

The present disclosure can also be described in accordance with the following numbered clauses.

Clause 1. A process for recovering one or more dialkyl terephthalates from a feedstock, comprising: exposing a feedstock composition comprising one or more tetramethylcyclobutanediol (TMCD)—containing polyesters to: i) ethylene glycol (EG), methanol, or both; and ii) a depolymerization catalyst, in a first reaction vessel under depolymerization conditions to provide a first mixture having a first solid component and a first liquid component, the first liquid component comprising one or more depolymerization products, wherein the depolymerization conditions comprise a temperature of from 150° C. to 260° C., and a pressure of from 1 atm (14.7 psig) to 102 atm (1500 psig), and a time period of from 0.5 hours to 10 hours; cooling at least a portion of the first mixture to a temperature below 150° C.; exposing at least a portion of the first liquid component to an alcohol composition and an alcoholysis catalyst under conditions including a temperature of from 23° C. to 70° C. and a pressure of from 1 atm to 2 atm for a time period of 0.5 hours to 5 hours to provide a second mixture, the second mixture comprising one or more dialkyl terephthalates; and separating at least a portion of the one or more dialkyl terephthalates by solid-liquid separation.

Clause 2. The process of clause 1, wherein, in the step of the exposing a feedstock composition comprising one or more tetramethylcyclobutanediol (TMCD)—containing polyesters to: i) ethylene glycol (EG), methanol, or both; and ii) a depolymerization catalyst, a weight ratio of: the EG, methanol, or both; to the one or more tetramethylcyclobutanediol (TMCD)—containing polyesters is in a range of 4:1 to 2:1.

Clause 3. The process of clauses 1-2, wherein, in the step of the exposing a feedstock composition comprising one or more tetramethylcyclobutanediol (TMCD)—containing polyesters to: i) ethylene glycol (EG), methanol, or both; and ii) a depolymerization catalyst, a weight ratio of: the EG to the methanol is in a range of 10:0 to 0:10.

Clause 4. The process of clauses 1-3, wherein a weight ratio of the alcohol composition to the one or more tetramethylcyclobutanediol (TMCD)—containing polyesters is in a range of 2:1 to 10:1.

Clause 5. The process of clauses 1-4, wherein the feedstock composition comprises one or more foreign materials.

Clause 6. The process of clause 5, wherein the one or more foreign materials comprises at least one member selected from the group consisting of polyesters other than TMCD-containing polyesters and polyethylene terephthalate (PET), polyvinyl chloride (PVC), polyvinyl acetal, polyvinyl butyral (PVB), polyvinyl alcohol (PVOH), ethylene vinyl alcohol (EVOH), cotton, polyolefins, polystyrene, polycarbonate, Spandex, natural fibers, cellulose ester, polyacrylates, polymethacrylate, and polyamides, poly(lactic acid), polydimethylsiloxane, polysilane, calcium carbonate, titanium dioxide, inorganic fillers, dyes, pigments, plasticizers, adhesives, flame retardants, metals, aluminum, and iron.

Clause 7. The process of clauses 5-6, wherein the one or more foreign materials are present in an amount of from 0.01 wt. % to 50 wt. %, relative to the weight of the one or more TMCD-containing polyesters in the feedstock composition.

Clause 8. The process of clauses 1-7, wherein the depolymerization catalyst comprises a member selected from the group consisting of Li₂CO₃, CaCO₃, Na₂CO₃, Cs₂CO₃, ZrCO₃, LiOH, NaOH, KOH, tetrabutylammonium hydroxide (TBAH), sodium methoxide (NaOMe), lithium methoxide (LiOMe), magnesium methoxide (Mg(OMe)₂, potassium t-butoxide, ethylene glycol monosodium salt, ethylene glycol disodium salt, tetra isopropyl titanate (TIPT), 1,8-Diazabicyclo[5.4.0]undec-7-ene (DBU), zinc acetylacetonate hydrate (Zn(acac)₂), zinc acetate (Zn(OAc)₂), manganese (II) acetate (Mn(OAc)₂), hydrotalcite, zeolite, and lithium chloride.

Clause 9. The process of clause 8, wherein the depolymerization catalyst is NaOMe, Zn(OAc)₂, or both.

Clause 10. The process of clauses 1-9, wherein the depolymerization catalyst is present in an amount of from 0.1 wt. % to 10 wt. %, relative to the weight of the feedstock composition.

Clause 11. The process of clauses 1-10, wherein, the alcoholysis catalyst is present in an amount of from 0.1 wt. % to 20 wt. %, relative to the weight of the feedstock composition.

Clause 12. The process of clause 11, wherein the alcoholysis catalyst comprises K₂CO₃, Na₂CO₃, Li₂CO₃, Cs₂CO₃; KOH, LiOH, NaOH; NaOMe, Mg(OMe)₂, KOMe, KOt-Bu, ethylene glycol monosodium salt, ethylene glycol disodium salt, or a combination thereof.

Clause 13. The process of clauses 1-12, wherein the one or more depolymerization products comprise monomers, oligomers, or a combination thereof.

Clause 14. The process of clause 13, wherein the oligomers exhibit a degree of polymerization of from 2 to 10.

Clause 15. The process of clauses 1-14, further comprising: prior to the exposing at least a portion of the first liquid component to an alcohol composition, separating at least a portion of the first liquid component from the first solid component in the first mixture.

Clause 16. The process of clause 15, wherein the separating occurs at a temperature of from 50° C. to 150° C.

Clause 17. The process of clauses 5-7, wherein at least a portion of the foreign materials are present in the first solid component of the first mixture.

Clause 18. The process of clauses 1-17, wherein the second mixture comprises a second liquid component and a second solid component, and wherein, during the separating at least a portion of the one or more dialkyl terephthalates by solid-liquid separation, the one or more dialkyl terephthalates are present in the second solid component.

Clause 19. The process of clause 18, wherein the second liquid component comprises methanol, EG, TMCD, or a combination thereof.

Clause 20. The process of clauses 18-19, further comprising separating at least a portion of EG, methanol, TMCD, or a combination thereof, from the second mixture.

Clause 21. The process of clauses 1-20, wherein the feedstock further comprises polyethylene terephthalate (PET).

Clause 22. The process of clause 21, wherein a weight ratio of the PET relative to the TMCD-containing polyester in the feedstock composition is 10:1 to 1:10.

Clause 23. The process of clauses 1-22, wherein the alcohol composition comprises methanol.

Clause 24. The process of clauses 1-23, wherein the one or more dialkyl terephthalates comprises dimethyl terephthalate (DMT), and wherein the separating at least a portion of the one or more dialkyl terephthalates by solid-liquid separation comprises separating at least a portion of the DMT.

Clause 25. The process of clause 24, wherein the at least a portion of the DMT is at least 90% pure.

Clause 26. The process of clauses 15-16, wherein the separating comprises filtration, centrifugation, settling, sedimentation, or a combination thereof.

Clause 27. The process of clauses 1-26, wherein the process is conducted as a batch process, a semi-continuous process, or a continuous process.

This disclosure has been described in detail with particular reference to specific aspects thereof, but it will be understood that variations and modifications can be made within the spirit and scope of this disclosure.

1. A process for recovering one or more dialkyl terephthalates from a feedstock, comprising: exposing a feedstock composition comprising one or more tetramethylcyclobutanediol (TMCD)—containing polyesters to: i) ethylene glycol (EG), methanol, or both; and ii) a depolymerization catalyst, in a first reaction vessel under depolymerization conditions to provide a first mixture having a first solid component and a first liquid component, the first liquid component comprising one or more depolymerization products, wherein the depolymerization conditions comprise a temperature of from 150° C. to 260° C., and a pressure of from 1 atm (14.7 psig) to 102 atm (1500 psig), and a time period of from 0.5 hours to 10 hours; cooling at least a portion of the first mixture to a temperature below 150° C.; exposing at least a portion of the first liquid component to an alcohol composition and an alcoholysis catalyst under conditions including a temperature of from 23° C. to 70° C. and a pressure of from 1 atm to 2 atm for a time period of 0.5 hours to 5 hours to provide a second mixture, the second mixture comprising one or more dialkyl terephthalates; and separating at least a portion of the one or more dialkyl terephthalates by solid-liquid separation.

2. The process of claim 1, wherein, in the step of the exposing a feedstock composition comprising one or more tetramethylcyclobutanediol (TMCD)—containing polyesters to: i) ethylene glycol (EG), methanol, or both; and ii) a depolymerization catalyst, a weight ratio of: the EG, methanol, or both; to the one or more tetramethylcyclobutanediol (TMCD)—containing polyesters is in a range of 4:1 to 2:1.

3. The process of claim 1, wherein, in the step of the exposing a feedstock composition comprising one or more tetramethylcyclobutanediol (TMCD)—containing polyesters to: i) ethylene glycol (EG), methanol, or both; and ii) a depolymerization catalyst, a weight ratio of: the EG to the methanol is in a range of 10:0 to 0:10.

4. The process of claim 1, wherein a weight ratio of the alcohol composition to the one or more tetramethylcyclobutanediol (TMCD)—containing polyesters is in a range of 2:1 to 10:1.

5. The process of claim 1, wherein the feedstock composition comprises one or more foreign materials, and wherein the one or more foreign materials comprises at least one member selected from the group consisting of polyesters other than TMCD-containing polyesters and polyethylene terephthalate (PET), polyvinyl chloride (PVC), polyvinyl acetal, polyvinyl butyral (PVB), polyvinyl alcohol (PVOH), ethylene vinyl alcohol (EVOH), cotton, polyolefins, polystyrene, polycarbonate, Spandex, natural fibers, cellulose ester, polyacrylates, polymethacrylate, and polyamides, poly(lactic acid), polydimethylsiloxane, polysilane, calcium carbonate, titanium dioxide, inorganic fillers, dyes, pigments, plasticizers, adhesives, flame retardants, metals, aluminum, and iron.

6. The process of claim 5, wherein the one or more foreign materials are present in an amount of from 0.01 wt. % to 50 wt. %, relative to the weight of the one or more TMCD-containing polyesters in the feedstock composition.

7. The process of claim 1, wherein the depolymerization catalyst comprises a member selected from the group consisting of Li₂CO₃, CaCO₃, Na₂CO₃, Cs₂CO₃, ZrCO₃, LiOH, NaOH, KOH, tetrabutylammonium hydroxide (TBAH), sodium methoxide (NaOMe), lithium methoxide (LiOMe), magnesium methoxide (Mg(OMe)₂, potassium t-butoxide, ethylene glycol monosodium salt, ethylene glycol disodium salt, tetra isopropyl titanate (TIPT), 1,8-Diazabicyclo[5.4.0]undec-7-ene (DBU), zinc acetylacetonate hydrate (Zn(acac)₂), zinc acetate (Zn(OAc)₂), manganese (II) acetate (Mn(OAc)₂), hydrotalcite, zeolite, and lithium chloride.

8. The process of claim 1, wherein the depolymerization catalyst is present in an amount of from 0.1 wt. % to 10 wt. %, relative to the weight of the feedstock composition.

9. The process of claim 7, wherein the depolymerization catalyst is NaOMe, Zn(OAc)₂, or both.

10. The process of claim 1, wherein, the alcoholysis catalyst is present in an amount of from 0.1 wt. % to 20 wt. %, relative to the weight of the feedstock composition, and wherein the alcoholysis catalyst comprises K₂CO₃, Na₂CO₃, Li₂CO₃, Cs₂CO₃; KOH, LiOH, NaOH; NaOMe, Mg(OMe)₂, KOMe, KOt-Bu, ethylene glycol monosodium salt, ethylene glycol disodium salt, or a combination thereof.

11. The process of claim 1, wherein the one or more depolymerization products comprise monomers, oligomers, or a combination thereof, and wherein the oligomers exhibit a degree of polymerization of from 2 to 10.

12. The process of claim 1, further comprising: prior to the exposing at least a portion of the first liquid component to an alcohol composition, separating at least a portion of the first liquid component from the first solid component in the first mixture, and wherein the separating occurs at a temperature of from 50° C. to 150° C.

13. The process of claim 5, wherein at least a portion of the foreign materials are present in the first solid component of the first mixture.

14. The process of claim 1, wherein the second mixture comprises a second liquid component and a second solid component, and wherein, during the separating at least a portion of the one or more dialkyl terephthalates by solid-liquid separation, the one or more dialkyl terephthalates are present in the second solid component.

15. The process of claim 14, wherein the second liquid component comprises methanol, EG, TMCD, or a combination thereof.

16. The process of claim 15, further comprising separating at least a portion of EG, methanol, TMCD, or a combination thereof, from the second mixture.

17. The process of claim 1, wherein the feedstock further comprises polyethylene terephthalate (PET).

18. The process of claim 17, wherein a weight ratio of the PET relative to the TMCD-containing polyester in the feedstock composition is 10:1 to 1:10.

19. The process of claim 1, wherein the alcohol composition comprises methanol.

20. The process of claim 1, wherein the one or more dialkyl terephthalates comprises dimethyl terephthalate (DMT), and wherein the separating at least a portion of the one or more dialkyl terephthalates by solid-liquid separation comprises separating at least a portion of the DMT, and wherein the at least a portion of the DMT is at least 90% pure.
